(19) United States
(12) Patent Application Publication
(10) Pub. No.: US 2009/0104274 A1
(43) Pub. Date: Apr. 23, 2009

(54) PROCESS OF MAKING MICROSPHERES

(76) Inventors: Ajay Khopade, Baroda (IN); Alex George, Baroda (IN); Subhas Balaram Bhowmick, Baroda (IN)

Correspondence Address: SUGHRUE MION, PLLC, 2100 PENNSYLVANIA AVENUE, N.W., SUITE 800, WASHINGTON, DC, 20037, US

(21) Appl. No.: 11/817,630

(22) PCT Filed: Mar. 1, 2006
(86) PCT No.: PCT/IN06/00065

§ 371 Date: Jun. 27, 2008

(30) Foreign Application Priority Data
Mar. 1, 2005 (IN) 231/MUM/2005
Sep. 26, 2005 (IN) 1182/MUM/2005

(51) Int. Cl. A61K 9/14; B01J 19/00

(52) U.S. Cl. 424/490; 424/489; 422/129

(57) ABSTRACT

A process for the manufacture of free-flowing uniformly sized micro spheres for the sustained release of therapeutically active ingredient comprising: a) preparing a first dispersed phase comprising a therapeutically active ingredient, a biodegradable polymer and an organic solvent; b) mixing the first dispersed phase with a aqueous phase to form an emulsion; c) spraying the emulsion into a vessel (20) equipped with organic solvent removal means; d) passing the suspension of micro spheres through screens and collecting a fractionated size of the microspheres on the surface of the screens; e) drying the microspheres wherein steps a to e are carried out without manual intervention, in equipment connected in series, substantially unexposed to the environment;

### FIELD OF THE INVENTION

The present invention relates to a novel process for the manufacture of microspheres and/or microcapsules of therapeutically active ingredients.

### BACKGROUND OF THE INVENTION

Processes for preparing microcapsules and microspheres typically involve the formation of at least one dispersed phase and a continuous phase, wherein the two phases are emulsified to obtain the microcapsules or microspheres. The dispersed phase typically contains the active ingredient. In the process for preparing microspheres, the dispersed phase will also typically include a polymer so that, upon solidification within the continuous phase, the dispersed phase becomes a microsphere. Microcapsules are similarly formed using multiple phases. In a typical practice, a water-in-oil-in-water (W/O/W) emulsion is formed with the external continuous aqueous phase containing a polymer. The polymer is caused to precipitate out of the external continuous aqueous phase onto the surface of a dispersed phase to form a capsule or wall thereon. Once the microcapsules or microspheres are produced, they are then formulated into a finished dosage form by admixing them with suitable pharmaceutical excipients.

The advantages of encapsulating biologically active ingredients in biocompatible, biodegradable wall-forming materials to provide sustained or delayed release, and/or to protect the active ingredient from degradation, are widely reported. A variety of encapsulation methods and manufacture of microparticles are also reported. One such widely used technique is the W/O/W triple emulsion technique in which the biologically active ingredient is dissolved/dispersed in an aqueous phase, and emulsified with an oil phase comprising an organic solvent, in which the polymer is dissolved. The primary water-in-oil (W/O) emulsion so formed is then further emulsified with an aqueous phase consisting of a surfactant to form a W/O/W triple emulsion. Removal of organic solvent from the emulsion droplets by means of solvent evaporation results in hardening of the microparticles. Solvent evaporation may be carried out by various processes such as heating at an elevated temperature, by applying vacuum, by reduction of pressure, by blowing inert gas on a suspension comprising the formed microspheres or microcapsules, and the various such processes.

A variety of methods are employed for the manufacture of the W/O/W triple emulsion, such as by using stirrers, agitators, in-line mixing assemblies, or other dynamic mixing techniques. An efficient mixing mechanism is very critical in determining the droplet size and distribution of the resultant emulsion, which will eventually harden to form microparticles. U.S. Pat. Nos. 5,945,126 and 6,534,094 teach the use of primary emulsion preparation using an in-line homogenizer in which the aqueous and the organic polymer phase from two separate feed tanks are fed into an in-line homogenizer simultaneously, at specified feed rates, and the resultant emulsion is transferred to a third tank, in which the solvent evaporation is carried out. Assemblies like these include multiple processing vessels which require high level of cleanliness and more manufacturing area to be kept clean, for example, at a very low air particulate levels for sterile operations. Therefore, the use of multiple processing vessels increases the risks of contamination of products such as microspheres and microcapsules, which are required to have low bioburden.

The prior art methods of drying the formed microspheres or microcapsules, before reconstitution has certain disadvantages, which are listed below:

a. The prior art methods of drying the microspheres or microcapsules are cumbersome and time consuming.

b. The prior art methods of drying also present a risk of incomplete removal of solvent from the microsphere or microcapsule manufacture.

c. The prior art methods allow analysis of solvent removal only after the last freeze-drying step, which leads to a risk of failure of a batch, and does not allow in-process checks for solvent, particle size and other parameters that would ultimately govern the product quality.

There is also a need to meet these objectives while addressing the problems of obtaining microspheres or microcapsules that are sterile. Therefore, there is a need also for a process where equipments are substantially unexposed to the environment and to man, i.e. manual intervention is kept at a minimum.

The process of the present invention tries to solve these problems encountered in preparing microspheres and microcapsules. The present invention provides means of improving the solvent removal process by increasing the available surface area, by spraying the emulsion in the form of fine droplets. The drying process of the invention provides microspheres or microcapsules which have better flow properties, low bulk density and no static charge. Also, the process of the present invention can provide a high level of assurance of sterility since the process is carried out with minimal manual intervention, regardless of whether the manufacturing is carried out in very large batches or small batches. In contrast, prior art processes, such as those in U.S. Pat. No. 5,945,126, are particularly meant for continuous operation to produce very large quantities. The bulk lyophilization used in the present invention has advantages of more efficient mixing of stabilizer with the microspheres or microcapsules than the prior art unit lyophilization in vials/containers. Hence, the microspheres or microcapsules of the present invention have better morphological characteristics and the process can be subject to better in-process quality controls.

### OBJECTS OF THE INVENTION

It is an object of the present invention to provide a novel process for the manufacture of microspheres and/or microcapsules comprising therapeutically active ingredients.

It is another object of the present invention to provide a novel process for the manufacture of microspheres and/or microcapsules which can be carried out without manual intervention.

It is yet another object of the present invention to provide a novel process for the manufacture of microspheres and/or microcapsules which can be carried out without substantial exposure to the environment.

### SUMMARY OF THE INVENTION

The present invention relates to process for manufacture of microspheres and microcapsules and provides in its various embodiments the following:

A. A process for the manufacture of free-flowing uniformly sized microspheres or microcapsules for the sustained release of therapeutically active ingredient, the process comprising:
a. preparing a first dispersed phase comprising a therapeutically active ingredient, a biodegradable polymer and an organic solvent;
b. mixing the first dispersed phase with an aqueous phase to form an emulsion;
c. spraying the emulsion into a vessel equipped with organic solvent removal means.
d. passing the suspension of microspheres or microcapsules through a first screen to remove large sized microspheres or microcapsules having a size greater than the mesh size of the first screen and then through a second screen to remove microspheres or microcapsules having a size smaller than the mesh size of the second screen, thereby collecting a fractionated size of the microspheres or microcapsules on the surface of the second screen;
e. drying the microspheres or microcapsules,

wherein steps a to e are carried out without manual intervention, in equipment connected in series, substantially unexposed to the environment.

B. A process as described in A above, wherein the drying step comprises lyophilization, freeze-drying, or air-drying the microspheres or microcapsules.

C. A process for the manufacture of a lyophilized composition for the sustained release of a therapeutically active ingredient, said process comprising:
(a) preparing microspheres or microcapsules comprising a therapeutically active ingredient;
(b) transferring a suspension comprising microspheres or microcapsules and a stabilizer into shallow freeze-drying container;
(c) subjecting the suspension to lyophilization and dry-powder filling the lyophilized composition into unit dose containers.

D. A process for the manufacture of a lyophilized composition for the sustained release of a therapeutically active ingredient, the process comprising:
(a) preparing a first dispersed phase comprising a therapeutically active ingredient, a biodegradable polymer and an organic solvent;
(b) mixing the first dispersed phase with an aqueous phase to form an emulsion;
(c) spraying the emulsion into a vessel equipped with organic solvent removal means to prepare a suspension of microspheres or microcapsules in a liquid vehicle;
(d) passing the suspension of microspheres or microcapsules through a first screen to remove large sized microspheres or microcapsules having a size greater than the mesh size of the first screen and then through a second screen to remove microspheres or microcapsules having a size smaller than the mesh size of the second screen, thereby collecting a fractionated size of the microspheres or microcapsules on the surface of the second screen;
(e) drying the microspheres or microcapsules;
(f) suspending the microspheres or microcapsules in aqueous solution of a stabilizer
(g) transferring the suspension comprising the microspheres or microcapsules and the stabilizer into shallow freeze-drying container;
(h) subjecting the suspension to lyophilization and dry-powder filling the lyophilized composition into unit dose containers,

wherein steps a to e are carried out without manual intervention, in equipment connected in series, substantially unexposed to the environment.

E. A process as described in D above, wherein the drying step comprises lyophilization, freeze-drying, or air-drying the microspheres or microcapsules.

F. A process as described in A above, wherein the first dispersed phase is a solution and the process produces microspheres.

G. A process as described in F above, wherein the microspheres comprise the therapeutically active ingredient uniformly distributed throughout a biodegradable polymer matrix.

H. A process as described in A above, wherein the first dispersed phase is an emulsion and the process produces microcapsules.

I. A process as described in H above, wherein the emulsion is prepared by a process comprising intermixing an aqueous solution comprising a therapeutically active ingredient with a solution of a biodegradable polymer in an organic solvent that is insoluble or only slightly soluble in water, in a first dispersed phase vessel.

J. A process as described in H above, wherein the first dispersed phase further comprises a retaining substance.

K. An apparatus for the manufacture of microspheres comprising:
(a) a first dispersed phase vessel for preparing a solution of the therapeutically active ingredient and biodegradable polymer in an organic solvent;
(b) means for forming an emulsion of the dispersed phase in an aqueous phase;
(c) a solvent evaporation vessel containing the emulsion and comprising a means for spraying the emulsion into the headspace of the vessel and a means for removing the organic solvent from the emulsion.

L. An apparatus as described in K above, wherein the means for removing the organic solvent is selected from applying vacuum, bubbling an inert gas through the emulsion, purging the head space of the solvent evaporation vessel with an inert gas, and a combination thereof

M. An apparatus for the manufacture of microcapsules comprising:
(a) a first dispersed phase vessel for preparing a first emulsion of the therapeutically active ingredient and biodegradable polymer in an organic solvent;
(b) means for forming a secondary emulsion of the first emulsion in an aqueous phase;
(c) a solvent evaporation vessel containing the second emulsion and comprising a means for spraying the secondary emulsion into the headspace of the vessel and a means for removing the organic solvent from the emulsion.

N. An apparatus as described in M above, wherein the means for removing the organic solvent is selected from applying vacuum, bubbling an inert gas through the emulsion, purging the head space of the solvent evaporation vessel with an inert gas, and a combination thereof.

### DESCRIPTION OF THE INVENTION

The present invention relates to a process for the manufacture of free-flowing, uniformly sized microspheres or microcapsules for the sustained release of therapeutically active ingredient, the process comprising:
a. preparing a first dispersed phase comprising a therapeutically active ingredient, a biodegradable polymer and an organic solvent;
b. mixing the first dispersed phase with an aqueous phase to form an emulsion;
c. spraying the emulsion into a vessel equipped with organic solvent removal means.
d. passing the suspension of microspheres or microcapsules through a first screen to remove large sized microspheres or microcapsules having a size greater than the mesh size of the first screen and then through a second screen to remove microspheres or microcapsules having a size smaller than the mesh size of the second screen, thereby collecting a fractionated size of the microspheres or microcapsules on the surface of the second screen;
e. drying the microspheres or microcapsules,

wherein steps a to e are carried out without manual intervention, in equipment connected in series, substantially unexposed to the environment.

The phrase “without manual intervention” as used herein means that the process is carried out mechanically with the help of equipments, which are arranged in series, so that the dispersed phase and the emulsions are pumped to the appropriate equipments for the different process steps without requiring any manual handling, thereby assuring minimal contamination of the product. This however, excludes in-process quality control checks, for example, sample withdrawal at various processing steps for analysis of parameters such as those ensuring complete emulsification, residual solvent content, and the like. The process of the present invention is carried out in equipments, which are arranged in series, in a manner that minimizes manual intervention, and which allows the process to be carried out substantially unexposed to the environment. The term “substantially unexposed to the environment” means that the equipment is closed to the environment during operation, except for ports necessarily required for operability. The process therefore results in microspheres or microcapsules that meet the stringent requirements of sterility.

The term “free-flowing microspheres or microcapsules” as used herein means microspheres or microcapsules that will flow adequately during powder filing into vials.

The term “therapeutically active ingredient” intends to include the active ingredient, optionally in combination with pharmaceutically acceptable carriers and, optionally additional ingredients such as antioxidants, stabilizing agents, permeation enhancers, and the like. The process of preparing microspheres or microcapsules of the present invention is particularly suited for use of peptides and/or proteins. Of particular interest are LH-RH agonists such as leuprolide, triptorelin, goserelin, nafarelin, historelin and buserelin, and salts thereof, LH-RH antagonists, somatostatin and its analogs such as octreotide, human calcitonin, salmon calcitonin and eel calcitonin, growth hormones, growth hormone releasing hormones, growth hormone releasing peptide, parathyroid hormones and related peptides, interferon, erythropoietin, GM-CSF, G-CSF, thymosin, antitrypsin, and enterostatin and the like.

The biodegradable polymers and their amounts used in the production of microspheres or microcapsules by the process of the present invention may vary depending upon desired clinical characteristics, such as biodegradability, which governs the release profile of the active ingredient, and biocompatibility. Not limitative, examples of the biodegradable polymers suitable for use in the present invention include cellulose acetate, cellulose acetate propionate, cellulose butyrate, cellulose propionate, cellulose valerate, cumaroneindene polymer, dibutylaminohydroxypropyl ether, ethyl cellulose, ethylene-vinyl acetate copolymer, glycerol distearate, hydroxypropylmethyl cellulose phthalate, 2-methyl-5-vinylpyridine methacrylate-methacrylic acid copolymer, polyamino acids, polyanhydrides, polycaprolactone, polycarbonate, polybutadiene, polyesters, aliphatic polyesters, polybutadiene, polyesters, polyhydroxybutyric acid, polymethyl methacrylate, polymethacrylic acid ester, polyolesters, polypropylene, polysaccharides such as alginic acid, chitin, chitosan, chondroitin, dextrin, dextran, hyaluronic acid, heparin, keratan sulfate, starch, polystyrene, polyvinyl acetal diethylamino acetate, polyvinyl acetate, polyvinyl alcohol, polyvinyl butyral, polyvinyl formal, proteins such as albumin, casein, collagen, fibrin, fibrinogen, gelatin, hemoglobin, transfferin, zein and so forth, vinylchloride-propylene-vinlyacetate copolymer, vinylchloride-vinylacetate polymer, waxes such as beef tallow, whale wax, bee wax, paraffin wax, castor wax and so forth, and higher lipid acids such as myristic acid, palmitic acid, stearic acid, behenic acid and so forth, with preference to aliphatic polyesters. Other polymers include homopolymers of lactic acid, and copolymers of lactic acid and glycolic acid, i.e., poly(lactide-co-glycolide) or polylactide or “PLGA” polymers, which includes polymers of lactic acid alone, copolymers of lactic acid and glycolic acid, mixtures of such polymers, mixtures of such copolymers, and mixtures of such polymers and copolymers—the lactic acid being either in racemic or in optically active form. The ratio of lactic acid residues to glycolic acid residues can vary, and typically ranges from 25:75 to 75:25, although even a 10% glycolide could find use since high lactide content results in lower viscosity and higher solubility. A homopolymer of lactic acid, or a copolymer of lactic acid and glycolic acid having a monomer ratio in the range of about 1:1 to about 3:1, may be used. The average molecular weight of the polylactide biodegradable polymer used may be about 5,000 to about 100,000 Daltons. The amount of the polymer that may be used in the process of the present invention depends upon the type of release desired from the microsphere or microcapsule, such as depending on whether the release is to be sustained for one month, two months, three months or six months, or more.

In the process of the present invention, the active ingredient is present in a first dispersed phase along with the biodegradable polymer and an organic solvent. Depending on the active ingredient and depending on the polymer that may be used, the first dispersed phase may be a solution or an emulsion. If the active ingredient is water-soluble, then it is typically dissolved in a minimal quantity of purified water, while the biodegradable polymer is dissolved in a suitable organic solvent. These two solutions are then emulsified to obtain the first dispersed phase. Alternatively, if the active ingredient is water-insoluble, then it is dissolved in the organic solvent along with the biodegradable polymer to obtain the first dispersed phase. In the process of the present invention, when the first dispersed phase used is a solution, then microspheres are produced by the process of the invention, whereas when the first dispersed phase used is an emulsion, microcapsules are produced by the process of the invention.

Solvents that may be used to dissolve the biodegradable polymer will depend upon a number of factors, including the nature of the polymer, the active agent that has to be encapsulated, toxicity of the solvent, compatibility with other solvents in the system and even the use to which the microsphere/microcapsule will be put. Thus, in addition to dissolving the polymer, the solvent must be immiscible with the continuous phase in order to form droplets (in cases where the dispersed phase is an emulsion), should be highly volatile for optimum evaporation efficiency, and should desirably be non-flammable for safety reasons. Solvents suitable for the process of the present invention include, but are not limited to, methylene chloride, chloroform, ethyl acetate, substituted pyrrolidone and the like and mixtures thereof. Typically, the solvents are used in the minimum required amount.

In cases where the dispersed phase is an emulsion, the active ingredient may be mixed with a active ingredient-retaining substance. The active ingredient retaining substance employed in accordance with the present invention is either a substance which is soluble in water and hardly soluble in the organic solvent contained in said oil layer and when dissolved in water assumes a viscous semi-solid consistency, or a substance which gains considerably in viscosity to provide a semi-solid or solid matrix under the influence of an external factor such as temperature, pH, metal ions (e.g. Cu++, Al+++, Zn++, etc.), organic acids (e.g. tartaric acid, citric acid, tannic acid, etc.), a salt thereof (e.g. calcium citrate, etc.), chemical condensing agents (e.g. glutaraldehyde, acetoaldehyde), and the like. Examples of such active ingredient-retaining substance include, among others, natural mucilages such as gum acacia, Irish moss, gum karaya, gum tragacanth, gum guaiac, gum xanthan, locust bean gum, and the like; synthetic mucilages, and high molecular weight compounds, which include various proteins such as casein, gelatin, collagen, albumin (e.g. human serum albumin), globulin, fibrin, and the like, and various carbohydrates such as cellulose, dextrin, pectin, starch, agar, mannan, and the like. These substances may be used as they are or in chemically modified forms, e.g. esterified or etherified forms (e.g. methylcellulose, ethylcellulose, carboxymethylcellulose, gelatin succinate, and the like), hydrolyzed forms (e.g. sodium alginate, sodium pectinate) or salts thereof. As examples of synthetic high molecular weight compounds may be mentioned polyvinyl compounds (e.g. polyvinyl pyrrolidone, polyvinyl alcohol, polyvinyl methyl ether, polyvinyl ether), polycarboxylic acids (e.g. polyacrylic acid, polymethacrylic acid, Carbopol [Goodrich & Co., U.S.A.]), polyethylene compounds (e.g. polyethylene glycol) and polysaccharides (e.g. polysucrose, polyglucose, polylactose) and salts thereof. Among the aforementioned compounds, gelatin, albumin, pectin and agar are particularly desirable. These compounds may be used alone or in combination and while the proportion of such compounds depends on the kind of compound, it is selected from the range of about 0.05% to about 80% (w/w) in terms of concentration in the first dispersed phase, preferably from the range of about 0.1% to about 50% (w/w) on the same basis. It should, however, be understood that such compounds must be used in sufficient amounts to ensure that the initial viscosity of the first dispersed phase in the W/O/W emulsion described hereinafter will be not lower than about 5000 centipoises (cps), preferably not lower than about 10000 cps, or the first dispersed phase may be increased in viscosity to not lower than about 5000 cps, preferably not lower than about 10000 cps, or be solidified by external factors.

The active ingredient-retaining substance may be dissolved along with the active ingredient in aqueous medium and emulsified with a water-immiscible organic solvent comprising the biodegradable polymer to obtain the first dispersed phase. Alternatively, the active ingredient and the active ingredient-retaining substance may be dissolved in a suitable solvent and subjected to lyophilization to obtain a sterile cake of the active ingredient and the active ingredient-retaining substance. This sterile cake is then used to prepare the first dispersed phase. Lyophilization to obtain the sterile cake improves the quality of the finished product, since lyophilization reduces the bioburden.

The process of the present invention involves preparing the first dispersed phase by (i) emulsifying an aqueous solution of the active ingredient, and optionally a active ingredient-retaining substance, with a solution of the biodegradable polymer in a suitable organic solvent that is immiscible with water, or (ii) by preparing a solution comprising the active ingredient and the biodegradable polymer in a suitable solvent. The process for manufacture of the first dispersed phase is carried out in a first tank, from which it is pumped to a second tank comprising the second phase, under aseptic conditions, and without any manual intervention.

The second phase is typically an aqueous solution of an emulsifying agent that assists in the formation of the final O/W or W/O/W emulsion. The second phase is prepared by simply dissolving the emulsifying agent in purified water under aseptic conditions. Examples of the emulsifying agents that may be used include, but are not limited to, anionic surfactants (e.g. sodium oleate, sodium stearate, sodium laurylsulfate, and the like), nonionic surfactants (e.g. polyoxyethylene sorbitan fatty acid esters [Tween 80 and Tween 60, Atlas Powder, U.S.A.], polyoxyethylene castor oil derivatives [HCO-60 and HCO-50, Nikko Chemicals, Japan], and the like), polyvinyl pyrrolidone, polyvinyl alcohol, carboxymethyl-cellulose, lecithin, gelatin, and the like. Such emulsifying agents may be used either alone or in combination. The concentration of the emulsifying agent may be selected from the range of about 0.01% to about 20% and is preferably in the range of about 0.05% to about 10%.

The first dispersed phase is pumped to the second tank containing the second aqueous phase, An in-line homogenizer that may be present at the base of the second tank aids in emulsification of the two phases to provide a W/O/W emulsion or a O/W emulsion, depending on whether the first dispersed phase is an emulsion or a solution. The emulsion thus formed is then pumped through one or more nozzles on the top of the tank using high throughput pumps. Pumping and spraying through these nozzles generates enormous surface area due to formation of minute droplets of the emulsion. The spraying thus results in very efficient solvent removal. The evaporation or removal of the solvent causes the emulsion droplets to harden into microspheres or microcapsules, as the case may be. The spraying apparatus (nozzles) may be suitably selected so as to control the size of the microspheres or microcapsules that are formed.

The spraying of the final W/O/W or the O/W emulsion for solvent evaporation is carried out in the second tank itself. Thus, in the process of the present invention, the second tank acts as the emulsification vessel, as well as the solvent evaporation vessel. The process of the present invention provides advantage over prior art assemblies which include multiple processing vessels requiring high level of cleanliness and more manufacturing area to be kept clean, for example, at a very low air particulate levels for sterile operations. Therefore, the process of the present invention lowers the risks of contamination of products.

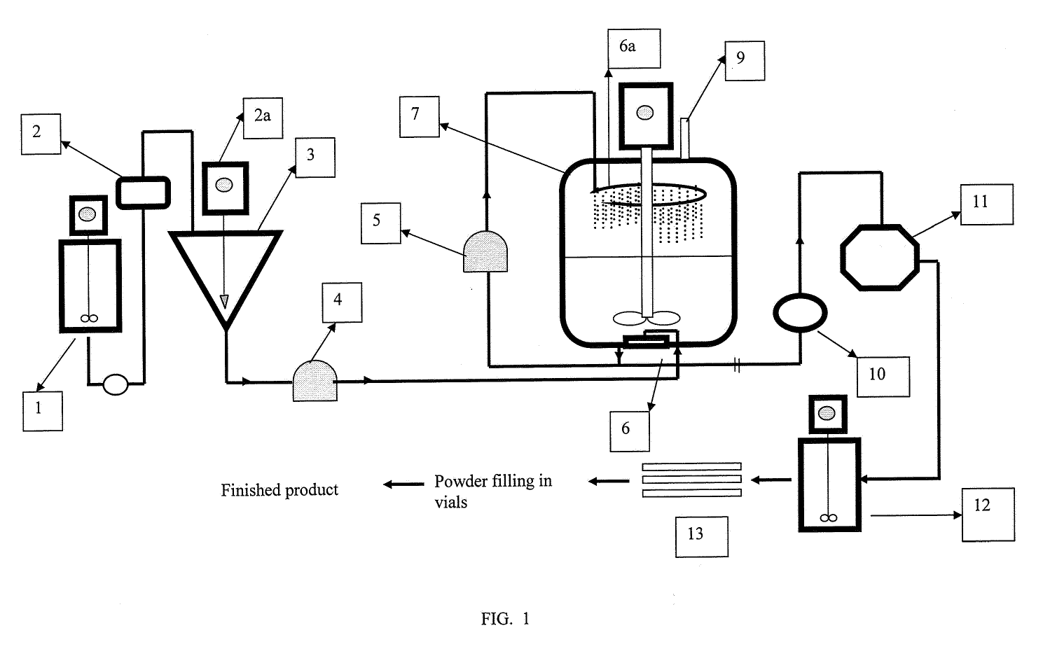

Spraying apparatus that may be used in the process of the present invention may be any of the typical apparatus used in the art, such as those described in embodiments and FIGS. 1-3 below, and are generically referred to herein as organic solvent removal means. The size of the microsphere or microcapsule depends on the spraying apparatus used, i.e. on the droplet size that would be produced upon spraying of the emulsion from the nozzles.

The process of the present invention typically provides microspheres/microcapsules having a volume mean diameter in the range of about 2 microns to about 200 microns, preferably about 10 microns to about 20 microns.

In one embodiment of the present invention, the primary emulsion comprising an aqueous phase with the active ingredient dissolved/dispersed therein, and the organic polymer phase prepared by stirring in a specially designed stainless steel tank, is fed into a tank containing the external aqueous phase and having an inbuilt homogenizer at the base of the tank. By controlling the homogenizer speed and the feed rate of the primary emulsion phase, the droplet size of the emulsion formed can be accurately controlled. It is highly important to control the microsphere/microcapsule size as it governs the release profile of the active ingredient.

In one embodiment of the present invention, the primary emulsion is pumped with the help of a high throughput pump (such as peristaltic pumps, diaphragm pumps) along with the external aqueous phase, through a jet spray nozzle. The jet spray nozzle has baffles incorporated therein, which act as a static mixer and results in the mixing and atomization of the two phases, thereby forming a triple emulsion.

In yet another embodiment of the present invention, with the help of a high throughput pump, the two phases are pumped through separate concentric tubings and are brought in contact at high speed and high shear conditions, resulting in the mixing and emulsification of the two phases.

The process of solvent evaporation may be enhanced by applying vacuum, bubbling an inert gas through the emulsion, purging the head space of the solvent evaporation vessel with an inert gas, and a combination thereof while spraying the emulsion through the nozzles. This helps in reducing the residual solvent content in the microspheres/microcapsules to levels far too low to cause any toxicity, and also reduce the time required for the process of solvent evaporation. Since the solvent evaporation process of the present invention is carried out in the emulsification vessel itself, and since there is no manual intervention at all, the process results in microspheres/microcapsules that have low bioburden.

We have observed that the dynamic transition temperature (dTg) of the biodegradable polymer used in the microspheres or microcapsules prepared by the process of the present invention plays a significant role in deciding the product characteristics. The dTg is the temperature above which, the secondary, non-covalent bonds between the polymer chains become weak in comparison to thermal motion, and the polymer becomes rubbery and capable of elastic or plastic deformation, without fracture. The dTg of the biodegradable polymer is low when the amount of residual solvent within the microspheres/microcapsules is high, and this dTg goes on increasing gradually as the solvent in the microspheres/microcapsules is gradually evaporated. It was surprisingly found that the temperature at which solvent evaporation is carried out can affect the physical as well as release characteristics of the microspheres/microcapsules. If the temperature is always maintained below the dTg of the biodegradable polymer at any given point during the process of solvent evaporation, and gradually increased to the dTg, microspheres/microcapsules with desirable physical properties and release profile could be obtained. However, if the solvent evaporation is carried out at a temperature above the dTg of the polymer, or increased to a temperature above the dTg of the polymer, the microspheres/microcapsules were found not to have good physical characteristics, and had a slow release profile, at times releasing a maximum of only 70% of the active ingredient.

In the process of the present invention, solvent evaporation causes the microspheres or microcapsules to harden. These hardened microspheres/microcapsules are collected from the bottom of the tank in the form of a suspension in the continuous aqueous phase. This suspension is then subjected to drying and fractionation on the basis of size to obtain uniformly sized microspheres/microcapsules. Typically, the drying and size separation operations are carried out in an apparatus that may be referred to as “microsphere/microcapsule fractionation and isolation unit”. Such a unit/apparatus has a first screen to remove microspheres or microcapsules having a size greater than the mesh size of the first screen, and a second screen to remove microspheres or microcapsules having a size smaller than the mesh size of the second screen, thereby allowing collection of a fractionated size of the microspheres or microcapsules on the surface of the second screen. The first and second screens may be selected on the basis of the size of the microspheres or microcapsules desired. Thus, the process of the present invention provides uniformly sized microspheres/microcapsules. The desired fraction of the microspheres or microcapsules is then subjected to drying within the same unit/apparatus by means of applying vacuum. This drying results in the formation of free-flowing microspheres/microcapsules. Commercially available apparatus such as PharmAsep available from Sweco Inc. may be used for such purposes.

Alternatively, the microsphere/microcapsule fractionation and isolation unit may be subjected to freeze-drying, i.e. in situ freeze-drying or lyophilization of the microspheres/microcapsules, which results in microspheres/microcapsules with better flow properties and better suspension properties. Such a freeze-drying prevents formation of agglomerates that may be formed during drying of the microspheres/microcapsules. The agglomerates are undesirable as they are difficult to disperse or suspend in pharmaceutical vehicles.

The present invention also relates to a process for the manufacture of a lyophilized composition for the sustained release of a therapeutically active ingredient, the process comprising:
a. preparing a first dispersed phase comprising a therapeutically active ingredient, a biodegradable polymer and an organic solvent;
b. mixing the first dispersed phase with an aqueous phase to form an emulsion;
c. spraying the emulsion into a vessel equipped with organic solvent removal means to prepare a suspension of microspheres or microcapsules in a liquid vehicle;
d. passing the suspension of microspheres or microcapsules through a first screen to remove large sized microspheres or microcapsules having a size greater than the mesh size of the first screen and then through a second screen to remove microspheres or microcapsules having a size smaller than the mesh size of the second screen, thereby collecting a fractionated size of the microspheres or microcapsules on the surface of the second screen;
e. drying the microspheres or microcapsules;
f. suspending the microspheres or microcapsules in aqueous solution of a stabilizer,
g. transferring a suspension comprising the microspheres or microcapsules and a stabilizer into shallow freeze-drying container;
h. subjecting the suspension to lyophilization and dry-powder filling the lyophilized composition into unit dose containers,

wherein steps a to e are carried out without manual intervention, in equipment connected in series, substantially unexposed to the environment.

As described above, the free-flowing microspheres/microcapsules obtained upon drying are further suspended in aqueous solution of a stabilizer. The term “stabilizer” as used herein may be used interchangeably with the term “cryoprotectant” and includes all excipients that provide protection during the lyophilization process, to which the suspension is eventually subjected. Examples of stabilizers include, but are not limited to, carbohydrates, lipophilic molecules (such as sterols and glycols) linked to molecules containing polyhydroxyl groups (such as carbohydrates) via hydrophilic groups (such as polyoxyethylene), glycerol compounds, propanediol, cystein, cysteinate HCl and the like. Use of cryoprotectants ensures that the microspheres/microcapsules do not undergo any degradation during the process of lyophilization, such as undesired agglomeration of the microspheres/microcapsules, and/or undesirable change in physical properties of the microspheres/microcapsules, which may affect release of the active ingredient. The stabilizer is used in amounts conventional to the art of lyophilization.

The suspension of the microspheres/microcapsules is subjected to the process of lyophilization. Such a lyophilization may be carried out in vials or containers by introducing a unit dose of the microsphere/microcapsule suspension in the vials/containers, i.e. unit lyophilization. Alternatively, the suspension may be subjected to bulk lyophilization wherein the suspension is poured into shallow trays and the trays are then kept in the lyophilizer. We have observed that such bulk lyophilization in shallow trays assures better quality of the microspheres/microcapsules because there is more uniform mixing of the microspheres/microcapsules with the stabilizer and therefore better cryoprotection during the lyophilization cycle. The chances of physical segregation of the stabilizer and the microspheres/microcapsules are very slight in the shallow trays. The lyophilized powder thus obtained may then be aseptically filled into suitable containers.

The process of manufacture of microspheres or microcapsules of the present invention is illustrated by embodiments described here in below.

FIG. 1 shows an embodiment of the process of making microspheres or microcapsules;

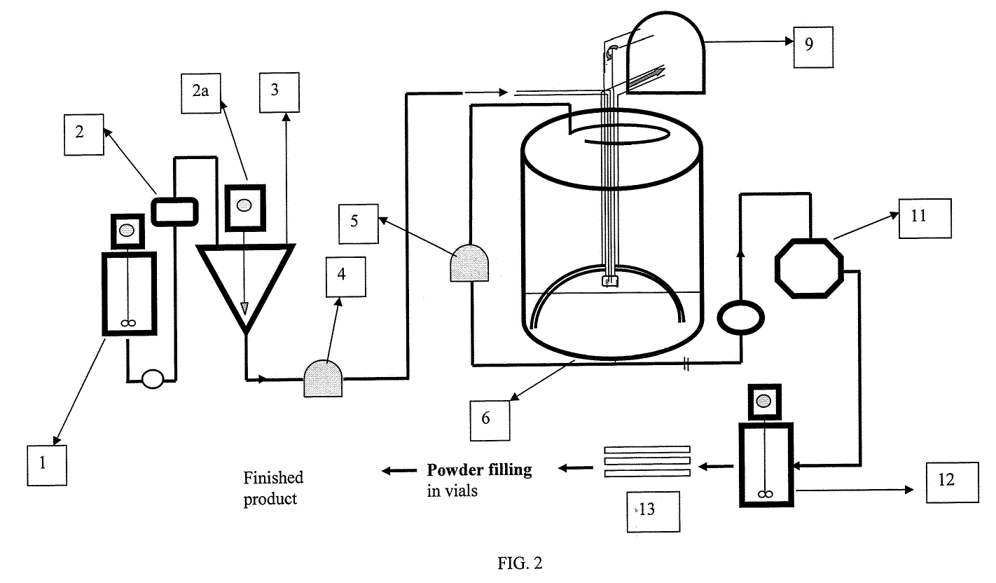

FIG. 2 shows another embodiment of the process of making microspheres or microcapsules;

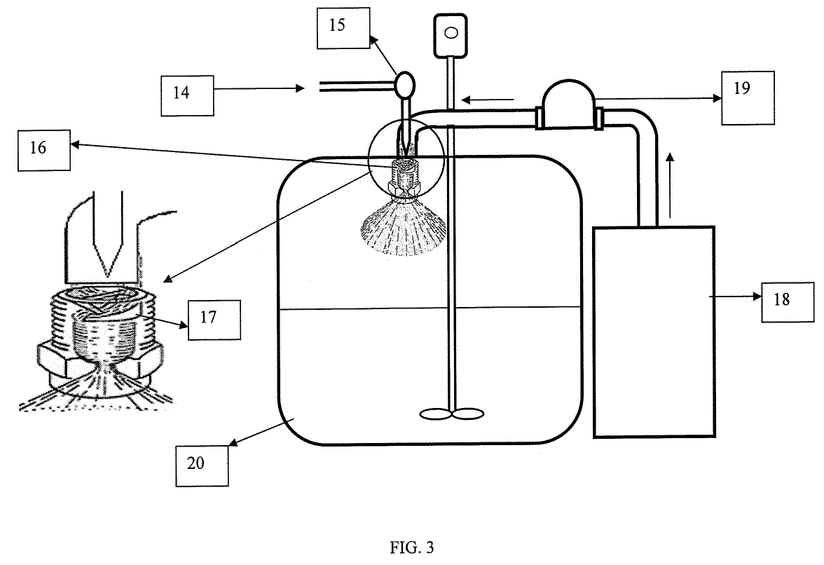

FIG. 3 shows yet another embodiment of the process of making microspheres or microcapsules.

Many aspects of the invention can be better understood with reference to the above drawings. The components in the drawings are not necessarily drawn to scale, emphasis instead being placed upon clearly illustrating the principles of the present invention.

FIG. 1

1: polymer dissolution vessel
2: Teflon capsule filter
2a: active ingredient dissolution vessel
3: first dispersed phase vessel
4: pump for feeding first dispersed phase
5: pump for spraying the microparticulate suspension
6: inbuilt inline homogenizer
6a: spraying assembly
7: microparticle formation cum solvent evaporation tank
9: to vacuum pump
10: pump
11: microsphere/microcapsule fractionation, isolation and bulk lyophilization unit
12: resuspension in stabilizer
13: bulk lyoplilization in sterile trays

FIG. 2

1: polymer dissolution vessel
2: Teflon capsule filter
2a: active ingredient dissolution vessel
3: first dispersed phase vessel
4: pump for feeding first dispersed phase
5: pump for spraying the microparticulate suspension
6: barrel mixing, dispensing system and solvent evaporation tank
9: pump to aid mixing of the dispersed phase with aqueous phase for microparticle formation
11: microsphere/microcapsule fractionation, isolation and bulk lyophilization unit
12: resuspension in stabilizer
13: bulk lyoplilization in sterile trays

FIG. 3

14: direction of pumping of first dispersed phase
15: pump to generate mist through jet spray nozzle
16: emulsification chamber
17: baffles in the emulsification chamber
18: container containing aqueous phase
19: pump
20: solvent evaporation vessel

In the embodiment illustrated in FIG. 1, following are the stages of the manufacturing process:

1. Sterilization of active ingredient and gelatin is done by combined dissolution in water for injection, filtration using a capsule filter (0.2 micron, Nylon 6,6) and lyophilization to get a combination cake.

2. The aqueous phase is prepared in an active ingredient dissolution vessel.

3. The polymer solution is prepared and filtered through PTFE capsule filter.

4. The solution/primary emulsion is prepared in first dispersed phase vessel.

5. PVA solution is prepared and filtered.

6. The emulsion/triple emulsion is prepared using a solvent evaporation vessel with inbuilt homogenizer at the base.

7. Homogenization process lasts till solution/primary emulsion is fed into the solvent evaporation vessel. After this step, homogenizer is switched off and solvent evaporation step is initiated.

8. Solvent evaporation is done under vacuum.

9. Wet sieving and drying is done using a microsphere/microcapsule fractionation and isolation unit. A vibro-filter dryer system such as the Sweco PharmASep (Sweco Inc., Florence, Ky.) may be used. This unit can be used for wet sieving of the microspheres/microcapsules and also for the in-situ lyophilization of the same. For this, the unit may have liquid nitrogen jacketing or bulk lyophilization capabilities.

10. In process analysis for assay/entrapment done.

11. Resuspension of microspheres/microcapsules in stabilizer is done.

12. Bulk lyophilization of microsphere suspension in stabilizer is done in sterile trays. Shallow autoclavable trays such as Lyoguard trays from, W. L. Gore & Company, USA may be used.

13. Powder filling of lyophilized microspheres in vials is done.

In the embodiment illustrated in FIG. 2, following are the stages of the manufacturing process:

1. Sterilization of active ingredient and gelatin is done by combined dissolution in water for injection, filtration using a capsule filter (0.2 micron, Nylon 6,6) and lyophilization to get a combination cake.

2. The aqueous phase is prepared in an active ingredient dissolution vessel.

3. The polymer solution is prepared and filtered through PTFE capsule filter.

4. The solution/primary emulsion is prepared in first dispersed phase vessel.

5. PVA solution is prepared and filtered.

6. The emulsion/triple emulsion is prepared using a barrel mixing and dispensing system with a hydrodynamic propeller provided at the base of the mixing chamber which rotates under shear from the flow of the liquids and further aids emulsification. 7. Solvent evaporation is done under vacuum.

8. Wet sieving and drying is done using a microsphere/microcapsule fractionation and isolation unit. A vibro-filter dryer system such as the Sweco PharnASep (Sweco Inc., Florence, Ky.) may be used. This unit can be used for wet sieving of the microspheres/microcapsules and also for the in-situ lyophilization of the same. For this, the unit may have liquid nitrogen jacketing or bulk lyophilization capabilities.

9. In process analysis for assay/entrapment done.

10. Resuspension of microspheres in stabilizer is done.

11. Bulk lyophilization of microsphere suspension in stabilizer is done in sterile trays.

12. Powder filling of lyophilized microspheres/microcapsules in vials is done.

In the embodiment illustrated in FIG. 3, following are the stages of the manufacturing process:

1. Sterilization of active ingredient and gelatin is done by combined dissolution in water for injection, filtration using a capsule filter (0.2 micron, Nylon 6,6) and lyophilization to get a combination cake.

2. The aqueous phase is prepared in an active ingredient dissolution vessel.

3. The polymer solution is prepared and filtered through PTFE capsule filter.

4. The solution/primary emulsion is prepared in first dispersed phase vessel.

5. PVA solution is prepared and filtered.

6. The emulsion/triple emulsion is prepared using a solvent evaporation vessel with a jet spray nozzle which has baffles located in its flow path This assembly acts like a static mixer, and in the resulting turbulence, emulsification takes place. The emulsion inlet port is so oriented that the dispersed phase is delivered at the point of turbulence and mixing with dispersion medium takes place instantly. The emulsion/triple emulsion so formed then passes out through the nozzle as fine droplets.

7. Wet sieving and drying is done using a microsphere/microcapsule fractionation and isolation unit. A vibro-filter dryer system such as the Sweco PharmASep (Sweco Inc., Florence, Ky.) may be used. This unit can be used for wet sieving of the microspheres/microcapsules and also for the in-situ lyophilization of the same. For this, the unit may have liquid nitrogen jacketing or bulk lyophilization capabilities.

8. In process analysis for assay/entrapment done.

9. Resuspension of microspheres in stabilizer is done.

10. Bulk lyophilization of microsphere suspension in stabilizer in sterile trays.

11. Powder filling of lyophilized microspheres in vials.

An important advantage of the setups mentioned in FIGS. 1 to 3 is that this enables the manufacture of microsphere/microcapsule formulations from 0.1 kg to 1.0 kg or above without changing the vessel dimensions. This is to be considered in the light that changes in equipment design or dimension in manufacturing set up is critical to the microspheres/microcapsules formed, in that the release properties may be changed significantly with such changes.

The process of the present invention tries to solve the problems encountered in preparing microspheres and microcapsules by the known prior art processes. The drying process of the invention provides microspheres or microcapsules having better flow properties, low bulk density and no static charge. Also, the microspheres or microcapsules of the invention are sterile as there is no manual intervention during steps a to e of the process. The bulk lyophilization used in the present invention has advantages of more efficient mixing of the stabilizer with the microspheres or microcapsules than the prior art unit lyophilization in vials/containers.

The microspheres/microcapsules obtained by the process of the present invention may then be admixed with pharmaceutical excipients conventional to the pharmaceutical art of parenteral dosage forms. The dosage form thus obtained are suitable for subcutaneous and/or intramuscular administration.

The examples that follow are provided as illustrations and do not limit the scope of the present invention.

### EXAMPLE 1

Microspheres of leuprolide acetate are prepared by the process given in Table 1 below.

| | | Quantity |
|---|---|---|
| Sr. No. | Materials | (mg) |
| 1. | Leuprolide Acetate | 20.000 |
| 2. | Gelatin | 3.46 |
| 3. | Copolymer of DL-Lactic acid and Glycolic acid | 176.54 |
| | (PLGA 7515) | |

### Method of Preparation:

Leuprolide acetate and gelatin are dissolved in about 20 ml of water for injection in the specially designed stainless steel tank at 50-60° C. The solution so prepared is kept under stirring and a solution comprising the polymer dissolved in 250 to 300 ml of methylene chloride is added. High speed stirring at 30-40° C. results in the formation of primary water in oil emulsion. The emulsion is allowed to stabilize for about 30 minutes at 15 to 20° C. This primary emulsion is then transferred with the help of a pump to a tank containing 100 L of 0.1% PVA solution stored at 15-20° C. through the inbuilt homogenizing assembly to produce triple emulsion. The emulsion is then subjected to solvent evaporation for 5 to 10 hours during which it is sprayed through multiple jet spray nozzles located at the top of the solvent evaporation tank alternatively accompanied with heating the solvent evaporation tank at a temperature below the dTg of the PLGA polymer and gradually raising the temperature as the solvent evaporates. The hardened microspheres thus obtained are separated by passing the suspension first through a 150 micron sieve to remove larger particles and then through a collection sieve of size 25 microns. The collected microspheres are then dried by lyophilizing them, so as to get free flowing microspheres. The microspheres so obtained are then reconstituted in mannitol solution, filled in trays, and subjected to lyophilization to obtain a free-flowing powder of the final product, which is aseptically filled into vials.

Although the invention has been described in terms of particular embodiments and applications, one of ordinary skill in the art, in light of this teaching, can generate additional embodiments and modifications without departing from the spirit of or exceeding the scope of the claimed invention. It should be emphasized that the above-described embodiments of the present invention, particularly any “preferred” embodiments, are merely possible examples of the invention of implementations, merely set forth for a clear understanding of the principles of the invention. Accordingly, it is to be understood that the drawings and descriptions herein are proffered by way of example to facilitate comprehension of the invention and should not be construed to limit the scope thereof.

1. A process for the manufacture of free-flowing uniformly sized microspheres or microcapsules for the sustained release of therapeutically active ingredient, the process comprising:
a. preparing a first dispersed phase comprising a therapeutically active ingredient, a biodegradable polymer and an organic solvent;
b. mixing the first dispersed phase with an aqueous phase to form an emulsion;
c. spraying the emulsion into a vessel equipped with organic solvent removal means.
d. passing the suspension of microspheres or microcapsules through a first screen to remove large sized microspheres or microcapsules having a size greater than the mesh size of the first screen and then through a second screen to remove microspheres or microcapsules having a size smaller than the mesh size of the second screen, thereby collecting a fractionated size of the microspheres or microcapsules on the surface of the second screen;
e. drying the microspheres or microcapsules,
wherein steps a to e are carried out without manual intervention, in equipment connected in series, substantially unexposed to the environment.

2. A process as claimed in claim 1, wherein the drying step comprises lyophilization, freeze-drying, or air-drying the microspheres or microcapsules.

3. A process for the manufacture of a lyophilized composition for the sustained release of a therapeutically active ingredient, said process comprising:
a. preparing microspheres or microcapsules comprising a therapeutically active ingredient;
b. transferring a suspension comprising microspheres or microcapsules and a stabilizer into shallow freeze-drying container;
c. subjecting the suspension to lyophilization and dry-powder filling the lyophilized composition into unit dose containers.

4. A process for the manufacture of a lyophilized composition for the sustained release of a therapeutically active ingredient, the process comprising:
a. preparing a first dispersed phase comprising a therapeutically active ingredient, a biodegradable polymer and an organic solvent;
b. mixing the first dispersed phase with an aqueous phase to form an emulsion;
c. spraying the emulsion into a vessel equipped with organic solvent removal means to prepare a suspension of microspheres or microcapsules in a liquid vehicle;
d. passing the suspension of microspheres or microcapsules through a first screen to remove large sized microspheres or microcapsules having a size greater than the microspheres or microcapsules having a size smaller than the mesh size of the second screen, thereby collecting a fractionated size of the microspheres or microcapsules on the surface of the second screen;
e. drying the microspheres or microcapsules;
f. suspending the microspheres or microcapsules in aqueous solution of a stabilizer,
g. transferring the suspension comprising the microspheres or microcapsules and the stabilizer into shallow freeze-drying container;
h. subjecting the suspension to lyophilization and dry-powder filling the lyophilized composition into unit dose containers,
wherein steps a to e are called out without manual intervention, in equipment connected in series, substantially unexposed to the environment

5. A process as claimed in claim 4, wherein the drying step comprises lyophilization, freeze-drying, or air-drying the microspheres or microcapsules.

6. A process as claimed in claim 1, wherein the first dispersed phase is a solution and the process produces microspheres.

7. A process as claimed in claim 6, wherein the microspheres comprise the therapeutically active ingredient uniformly distributed throughout a biodegradable polymer matrix.

8. A process as claimed in claim 1, wherein the first dispersed phase is an emulsion and the process produces microcapsules.

9. A process as claimed in claim 8, wherein the emulsion is prepared by a process comprising intermixing an aqueous solution comprising a therapeutically active ingredient with a solution of a biodegradable polymer in an organic solvent that is insoluble or only slightly soluble in water, in a first dispersed phase vessel.

10. A process as claimed in claim 8, wherein the first dispersed phase further comprises a n active ingredient-retaining substance.

11. An apparatus for the manufacture of microspheres comprising:
a. a first dispersed phase vessel for preparing a solution of the therapeutically active ingredient and biodegradable polymer in an organic solvent;
b. means for forming an emulsion of the dispersed phase in an aqueous phase;
c. a solvent evaporation vessel containing the emulsion and comprising a means for spraying the emulsion into the headspace of the vessel and a means for removing the organic solvent from the emulsion.

12. An apparatus as claimed in claim 11, wherein the means for removing the organic solvent is selected from applying vacuum, bubbling an inert gas through the emulsion, purging the head space of the solvent evaporation vessel with an inert gas, and a combination thereof.
